(19) United States
(12) Patent Application Publication
(10) Pub. No.: US 2011/0245777 A1
(43) Pub. Date: Oct. 6, 2011

(54) ABSORBENT CATHETER PAD

(75) Inventors: Rafael Andino, Grayson, GA (US); Christopher Brooks, Glen Head, NY (US)

(73) Assignee: Venetec International, Inc., Covington, GA (US)

(21) Appl. No.: 12/944,598

(22) Filed: Nov. 11, 2010

Related U.S. Application Data
(60) Provisional application No. 61/260,289, filed on Nov. 11, 2009.

(51) Int. Cl. A61M 25/02

(52) U.S. Cl. 604/180; 604/174

(57) ABSTRACT

A pad or medical article is configured to absorb fluids that are discharged from a catheter assembly and/or medical line. The pad includes a body member that can include a backing layer and an absorbent layer disposed over the backing layer. The absorbent layer can include a groove connecting the proximal end of the layer with a recess formed in a surface of the layer. The groove and recess can be configured to draw fluids that come into contact with the layer to the recess to evenly distribute the liquid throughout the absorbent layer. The medical article can also include a retention member disposed on the body member. The retention member can form a receiving space or channel so as to permit at least a portion of a catheter assembly or medical line to be placed within the receiving space.

### CROSS-REFERENCE TO RELATED APPLICATIONS

This application claims priority under 35 U.S.C. §119(e) to U.S. Provisional Application No. 61/260,289, filed Nov. 11, 2009, entitled “Absorbent Catheter Pad,” which is hereby incorporated by reference in its entirety.

### BACKGROUND

1. Field of the Invention

This invention relates to a medical article used to introduce a catheter into the vasculature of a patient. More particularly, this invention relates to a medical article including an absorbent layer for absorbing blood or other fluids that may discharge from a catheter and/or a medical line during introduction of the catheter into the patient.

2. Description of the Related Art

Health care providers routinely require access to the vasculature of a patient for delivery or withdrawal of fluids to or from the patient's bloodstream. When such access is required over any period of time, it is common to introduce a catheter or similar medical article into the bloodstream of the patient to provide reusable access, for instance in order to deliver medication and/or fluids directly into the bloodstream of the patient.

In intravenous applications, the catheter is generally short and includes a fitting, for example, a luer connector, at one end that is designed for attachment to another medical line or another medical article. Such a connector may also include a spin nut to lock the medical line to the catheter. In this way the same catheter may be connected to and released from different medical lines in order to exchange the medical lines without the need to introduce multiple intravenous catheters. In some cases, an extension set comprising a medical tube with a spin nut at one end is connected to the catheter so that the free end of the extension set can be attached to another medical line or system as desired, at a location further away from the insertion site.

When a needle of the catheter is introduced into the bloodstream of a patient, blood or other bodily fluids may be discharged from the connection end of the catheter before a medical line or another medical article can be attached to the catheter. Similarly, fluid from a medical line can be discharged from the medical line before the medical line can be attached to a catheter. Accordingly, these discharged fluids can come in contact with a patient's skin and/or a health care provider's skin.

### SUMMARY OF THE INVENTION

The devices and methods of the present invention have several features, no single one of which is solely responsible for its desirable attributes. Without limiting the scope of this invention as expressed by the claims which follow, its more prominent features will now be discussed briefly. After considering this discussion, and particularly after reading the section entitled “Detailed Description of Certain Embodiments,” one will understand how the features of this invention provide several advantages over other medical articles.

One aspect of the present invention is a pad including an absorbent layer and a retention member secured relative to the absorbent layer and defining a channel. The channel can be configured to releasably receive a portion of a medical article.

Another aspect of the present invention is a medical article for absorbing fluid during the introduction of a catheter into a patient. The medical article can include a body member having a backing layer defining a receiving space and can include an absorbent layer. At least a portion of the absorbent layer can be disposed in the receiving space. The medical article can also include a retention member secured relative to a proximal end of the body member and defining a channel. The channel can be configured to releasably receive at least a portion of another medical article.

Yet another aspect of the present invention is a method which includes providing a pad having an absorbent layer and a retention member secured relative to the absorbent layer, the retention member defining a channel. The method also includes placing at least a portion of a medical article within the channel such that the received portion of the medical article is secured relative to the pad.

### BRIEF DESCRIPTION OF THE DRAWINGS

FIG. 1 is a perspective view of a pad including a retention member according to a preferred embodiment of the present invention.

FIG. 2 is a perspective view of the pad from FIG. 1 with a catheter hub received within the retention member of the pad prior to insertion of the catheter into a patient. The catheter hub is engaged with a removable handpiece.

FIG. 3 is a perspective view of the pad from FIG. 2 with the catheter inserted into the patient and the handpiece disengaged from the catheter hub. The pad is disposed so that discharged fluid is absorbed by the pad.

FIG. 4 is a perspective view of the pad from FIG. 3 with the handpiece replaced by a spin nut of an extension set which is aligned with the catheter hub.

FIG. 5 is a perspective view of the spin nut of the extension set from FIG. 4 connected to the catheter hub with the pad being removed from the patient. The pad may be left on or removed from the patient.

### DETAILED DESCRIPTION OF CERTAIN EMBODIMENTS

The following description and the accompanying figures, which describe and show the preferred embodiments, are made to demonstrate several possible configurations that a medical article can take to include various aspects and features of the invention. Some of the illustrated embodiments are shown with an optional handpiece that can be utilized by a health care provider to facilitate insertion of a catheter needle or similar medical article into a patient to provide access to the patient's vasculature. The illustration of the medical article in this context is not intended to limit the disclosed aspects and features of the invention to the specified embodiments or to usage only with the illustrated handpiece. Those of skill in the art will recognize that the disclosed aspects and features of the invention are not limited to any particular application.

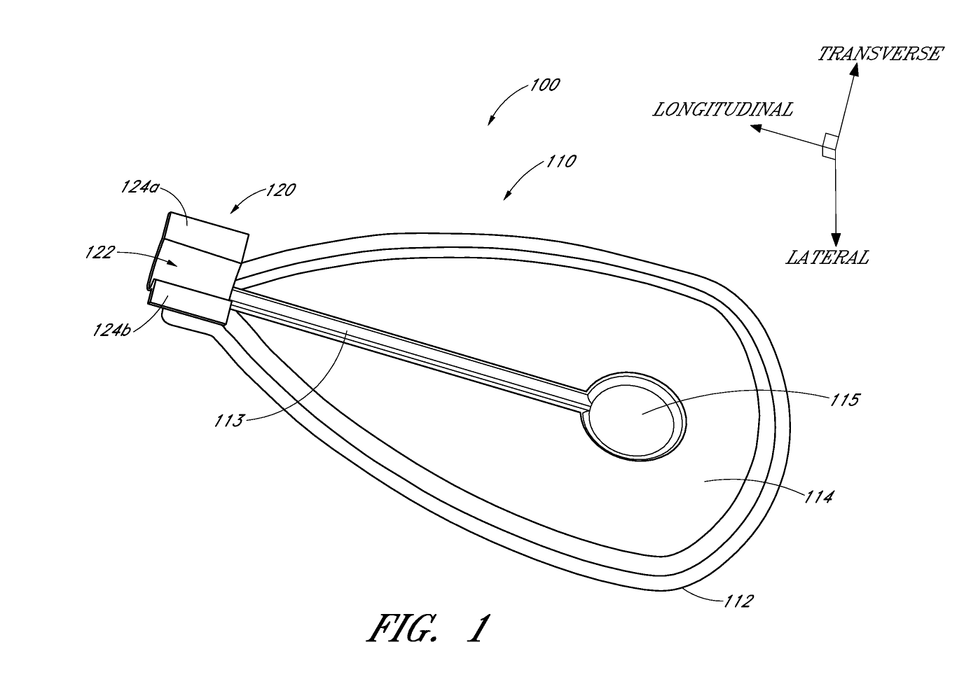

To assist with the description of the components of the absorbent pad or medical article, the following coordinate terms are used (see FIG. 1). A “longitudinal axis” is generally parallel to a portion of the absorbent pad, as well as generally parallel to a channel formed in the retention member or clip of the catheter pad. A “lateral axis” is normal to the longitudinal axis. A “transverse axis” extends normal to both the longitudinal and lateral axes. In addition, as used herein, “the longitudinal direction” refers to a direction substantially parallel to the longitudinal axis; “the lateral direction” refers to a direction substantially parallel to the lateral axis; and “the transverse direction” refers to a direction substantially parallel to the transverse axis. Also, the terms “proximal” and “distal”, which are used to describe the present medical article, are used consistently with the description of the exemplary applications (e.g., the illustrative examples of the use applications). Thus, proximal and distal are used in reference to the center of the patient's body. The terms “upper,” “lower,” “top,” “bottom,” “underside,” “upperside” and the like, which also are used to describe the present medical article, are used in reference to the illustrated orientation of the embodiment. For example, the term “upperside” is used to describe the portion of the medical article or catheter pad that is located above a lateral axis that passes through the longitudinal axis of the medical article. The term “underside” is used to describe the portion of the medical article that is located below a lateral axis that passes through the longitudinal axis of the medical article. Brief introductions to some of the features, which are common to the described embodiment of the catheter pad or medical article, are now described.

The preferred embodiments of the present invention advantageously provide a medical article for absorbing fluids that may be discharged from a catheter and/or medical line during the introduction of the catheter into a patient's vasculature or when a medical line is removed/replaced from the connection end of an inserted catheter. Thus, the medical article can act as a barrier to protect a patient and/or health care provider from any liquids that may be discharged during the initial insertion of the catheter or discharged when a medical article, such as an extension set, is disengaged from the catheter. For example, a first absorbent pad may be employed during the initial catheter insertion into the patient and then subsequently discarded after the first extension set is secured to the catheter. In between changing from the first extension set to a second extension set without removing the catheter from the patient, a second absorbent pad may be employed.

The medical article or pad preferably has a body member that comprises an absorbent layer configured to absorb fluids that contact the body member. The body member can include a backing layer configured to act as a barrier between the absorbent layer and the patient's skin. The medical article can also have a retention member for receiving a portion of the catheter hub. The retention member secures the received portion of the catheter hub relative to the body member. For example, the catheter hub can be inhibited or prevented from moving laterally, transversely, and/or longitudinally relative to the body member by the retention member. In the illustrated embodiment the retention member is disposed at a proximal end of the body member. The retention member can have a clip shape forming a channel which receives the portion of the catheter hub. The channel secures the catheter assembly relative to the body member of the catheter pad. Of course the location of the retention member on the body member is not limited to the illustrated location. Further, the retention member is not limited to the illustrated clip shape.

To facilitate a complete understanding of the illustrated embodiment, the remainder of the detailed description describes the medical article with reference to the attached figures, wherein like elements among the embodiments are referenced with like numerals throughout the following description.

FIG. 1 shows a perspective view of a medical article or pad 100 comprising a body member 110 and a retention member 120 coupled to the body member 110. The shape of the body member 110 can vary along the longitudinal axis between the most distal end and the most proximal end of the body member 110. For example, the body member 110 can be generally teardrop shaped.

The body member 110 includes an absorbent layer 114 and may further include a backing layer 112. In embodiments having a backing layer 112, preferably the backing layer 112 is disposed below at least a portion of the absorbent layer 114. The absorbent layer 114 can comprise any suitable absorbent material, for example, hydrophilic polymers, woven materials, sponges, foams, fabrics, organic materials, inorganic materials, and clay. In some embodiments, the absorbent layer 114 comprises a pad from an absorbent material.

The absorbent layer 114 can include a groove 113 disposed on a surface of the absorbent layer 114. The groove 113 can be extend between a proximal end of the absorbent layer 114 and a recess 115 disposed on the same side of the absorbent layer 114 as the groove 113. The groove 113 and recess 115 can be configured to draw or direct liquid that comes into contact with the absorbent layer 114 towards the recess 115 in order to evenly distribute the liquid throughout the absorbent layer 114. For example, the groove 113 may be angled relative to a bottom surface of the pad so as to facilitate fluid flow to the recess 115. In some embodiments, the recess 115 can be disposed near the geometric center of the absorbent layer 114 such that fluid received in the recess 115 is absorbed evenly throughout the absorbent layer 114.

The backing layer 112 acts as a barrier to confine liquid that contacts the absorbent layer 114 to the absorbent layer. Accordingly, the backing layer 112 can comprise any suitable material(s) that is liquid impermeable and/or that resists the absorption of liquids, for example, hydrophobic materials, non-woven materials, polypropylene, and polyethylene. In some embodiments, the backing layer 112 comprises a material that is more rigid than the absorbent layer 114 and has a concave shape to receive and house the absorbent layer 114 such that the absorbent layer 114 and backing layer 112 are secured relative to one another. The concave shape of the backing layer 112 can also act to pool and collect liquid that has been absorbed by the absorbent layer 114. The backing layer 112 can also comprise an adhesive layer disposed on at least a portion of a surface of the backing layer 112. In some embodiments, an adhesive layer is disposed on the surface of the backing layer 112 that is opposite the absorbent layer 114. The adhesive layer can be configured to releasably secure the backing layer 112 to another object, for example, to a patient.

Still referring to FIG. 1, a retention member 120 is disposed over a portion of the body member 110. The retention member 120 can be configured to form a receiving space or channel 122 therein to receive a portion of one or more other medical articles, for example, a catheter assembly or a handpiece. The retention member 120 can be configured to releasably secure another medical article, such as a catheter hub, relative to the catheter pad 100. In this way, the received portion of the medical article can be secured relative to a patient when the body member is releasably secured to a patient. In some embodiments, the retention member 120 comprises a U-shaped clip that has a longitudinal length sufficient to provide stability to a portion of a medical article that is received within the receiving space 122. The retention member 120 can further include tabs 124a, 124b to enable a healthcare provider to enlarge and restrict the receiving space 122 in order to receive and/or release a medical article therefrom. In some embodiments, the retention member 120 is integral with the backing layer 112. In other embodiments, the retention member is affixed to the body member 110.

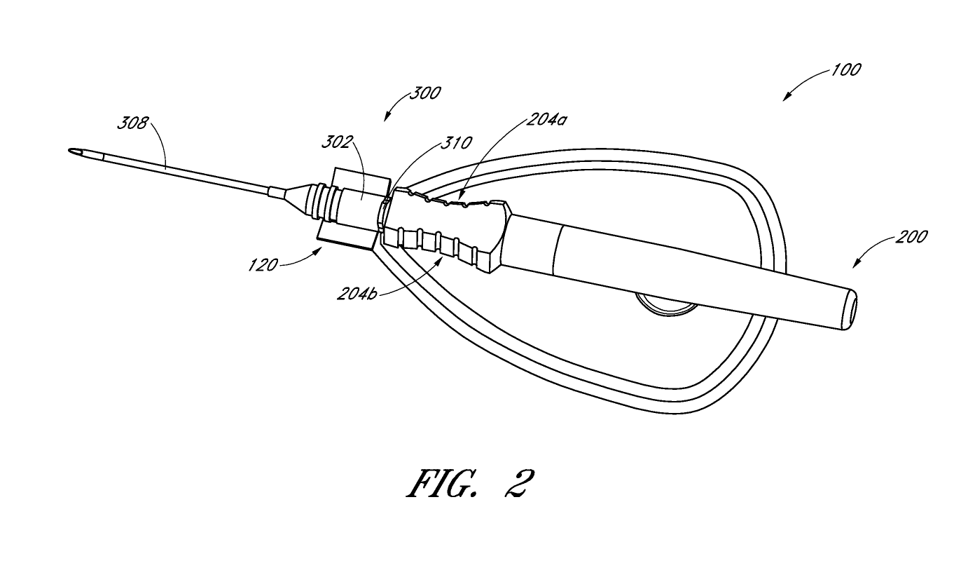

FIG. 2 shows a perspective view of the medical article or pad 100 from FIG. 1, along with a catheter assembly 300 and a handpiece or catheter introducer 200. The catheter assembly 300 includes an elongated body or catheter hub 302, a cannula or catheter 308 extending from the body 302 in a direction substantially parallel to the longitudinal axis of the catheter assembly 300, and a fitting 310. A central lumen or passageway through the catheter assembly 300 provides access to a patient's vasculature, for example, to withdraw blood and/or to introduce matter into the patient. The fitting 310 can comprise a luer connector or another fitting configured to connect the catheter assembly 300 with a variety of other medical articles including, medical lines, extension sets, other connector fittings, and the like.

As shown in FIG. 2, the fitting 310 is engaged with handpiece 200. In some embodiments, the catheter assembly 300 and handpiece 200 can be slidably engaged such that the catheter assembly 300 is partially inserted into a portion of handpiece 200. In some embodiments, the fitting 310 can abut the handpiece 200. The handpiece 200 can include one or more contoured surfaces 204a, 204b to facilitate proper gripping of the handpiece 200. By such a configuration, the handpiece 200 and the elongated body 302 can cooperate to assist a healthcare provider in handling and placing the catheter 308 in a patient's vasculature. A portion of the elongated body 302 can be received within the retention member 120 to releasably secure the catheter assembly 300 relative to the medical article or pad 100. At least a portion of the pad or medical article 100 is disposed underneath a portion of a junction between the handpiece 200 and the catheter assembly 300 so as to act as a barrier between the patient and blood or other leaked fluids.

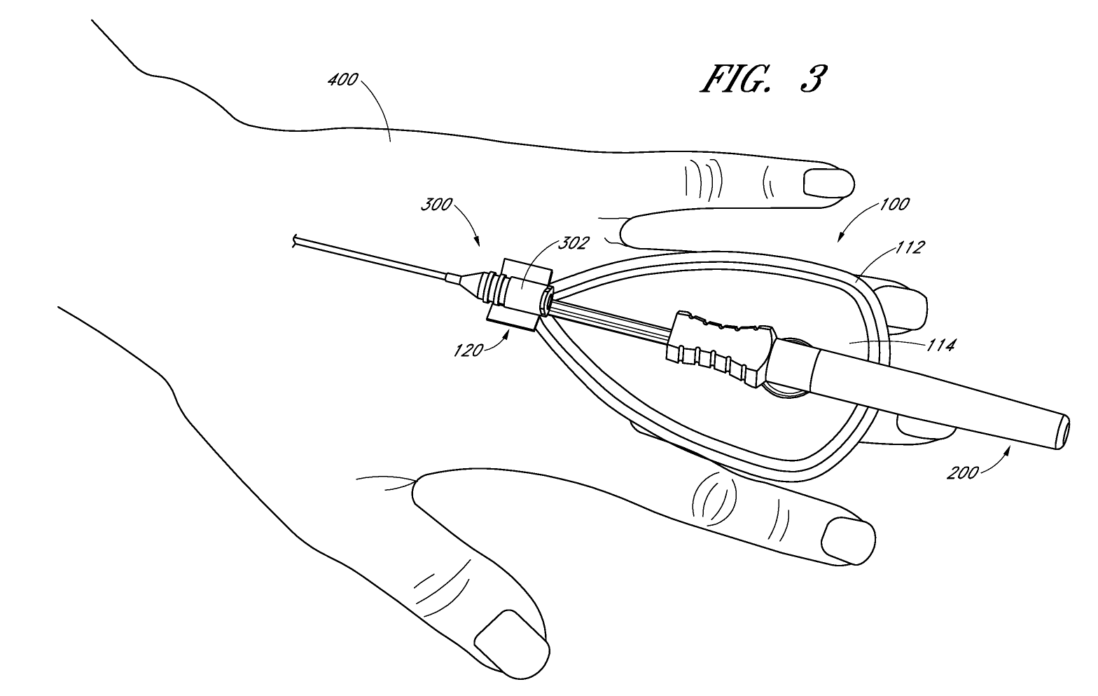

Turning now to FIG. 3, the catheter assembly 300 of FIG. 2 is shown inserted into a patient 400 with the handpiece 200 disengaged from the catheter assembly. Typically, after a catheter is introduced into a patient, fluid(s), for example, blood, may be discharged from the catheter hub 302 before a medical line is attached to a distal end of the catheter hub. The discharged fluid can contact the patient and/or health care provider's skin and present various problems. Because the catheter hub 302 shown in FIG. 3 is secured relative to the medical article 100 by the retention member 120, fluid discharged from the catheter hub 302 is absorbed by the absorbent layer 114. Furthermore, fluid that comes into contact with the absorbent layer 114 is confined thereto by the backing layer 112. Thus, the pad 100 protects the patient 400 and/or the healthcare provider introducing the catheter assembly 300 from discharged fluid.

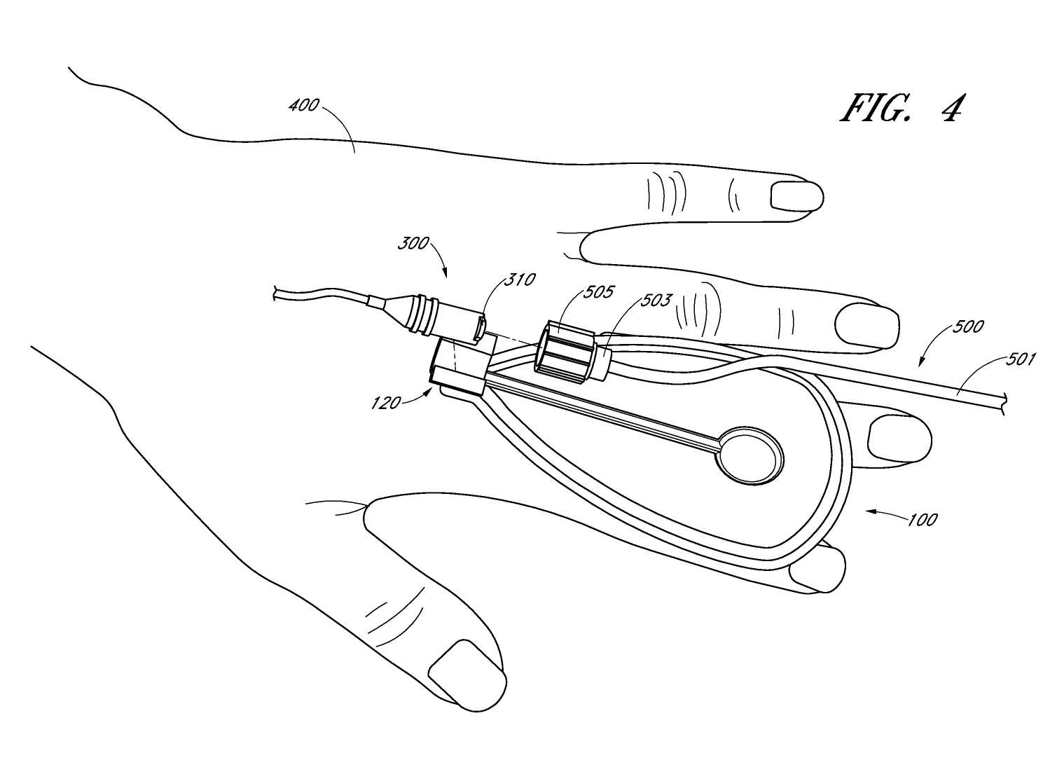

FIG. 4 shows the medical article 100 and catheter 300 from FIG. 3 with the catheter removed from the retention member 120. Also shown is an extension set 500 including a connector 503 and a medical line 501. The connector 503 is configured to connect the extension set 500 with the catheter assembly 300 to establish fluid communication therebetween. The connector 503 can include a spin nut 505 configured to engage the fitting 310 on the catheter hub 302 and releasably secure the extension set 500 relative to the catheter assembly 300. While the extension set 500 is being connected to the catheter assembly 300, the medical article 100 can be positioned on the patient 400 between the distal end of the catheter assembly and the proximal end of the extension set 500. With the medical article or pad 100 in such a position, the medical article may absorb fluid that is discharged from the catheter assembly 300 and/or extension set 500 and prevents the discharged fluid from contacting the patient 400 and or healthcare provider.

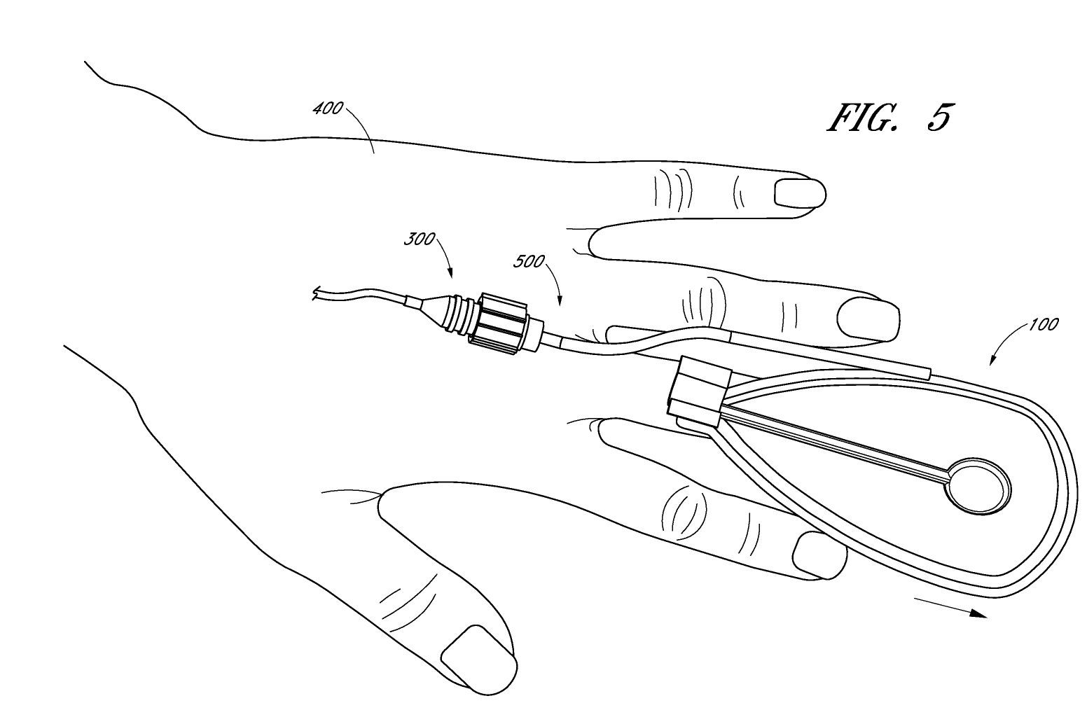

Turning now to FIG. 5, the catheter assembly 300 of FIGS. 2-4 is shown inserted into the patient 400 and connected to the extension set 500. After this connection has been made, the medical article 100 can be removed and disposed of. In instances where discharged fluid contacted the medical article 100, special care should be taken to properly dispose of the contaminated medical article 100.

The various embodiments of medical articles and techniques described above thus provide a number of ways to limit the exposure of fluids discharged from a catheter and/or medical line. In addition, the techniques and devices described may be broadly applied for use with a variety of medical articles and a variety of medical procedures.

Of course, it is to be understood that not necessarily all such objectives or advantages may be achieved in accordance with any particular embodiment using the systems described herein. Thus, for example, those skilled in the art will recognize that the systems may be developed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Although these techniques and systems have been disclosed in the context of certain embodiments and examples, it will be understood by those skilled in the art that these techniques and systems may be extended beyond the specifically disclosed embodiments to other embodiments and/or uses and obvious modifications and equivalents thereof. Additionally, it is contemplated that various aspects and features of the invention described can be practiced separately, combined together, or substituted for one another, and that a variety of combination and subcombinations of the features and aspects can be made and still fall within the scope of the invention. Thus, it is intended that the scope of the systems disclosed herein disclosed should not be limited by the particular disclosed embodiments described above but by a fair reading of the claims that follow.

What is claimed is:

1. A pad comprising:
an absorbent layer; and
a retention member secured relative to the absorbent layer and defining a channel, the channel being configured to releasably receive a portion of a medical article.

2. The pad of claim 1, wherein the retention member comprises a clip.

3. The pad of claim 2, wherein the clip comprises at least one tab configured to enable a healthcare provider to enlarge or restrict a dimension of the channel.

4. The pad of claim 1 further comprising a backing layer disposed underneath at least a portion of the absorbent layer.

5. The pad of claim 4, wherein the backing layer comprises a material that resists the absorption of liquids.

6. The pad of claim 4, wherein the backing layer forms a concave shape that receives at least a portion of the absorbent layer.

7. The pad of claim 4, wherein the retention member is integral with the backing layer.

8. The pad of claim 4, wherein the retention member is disposed near a proximal end of the absorbent layer.

9. The pad of claim 1 further comprising a groove disposed on a surface of the pad and generally aligned with a longitudinal axis of the pad so as to provide a flow path across at least a portion of the pad.

10. The pad of claim 9 further comprising a recess in the pad, the recess being fluidly coupled to the groove.

11. The pad of claim 10, wherein the groove is disposed on a surface of the absorbent layer.

12. A medical article for absorbing fluid during the introduction of a catheter into a patient, the medical article comprising:
a body member comprising
a backing layer defining a receiving space, and
an absorbent layer, at least a portion of the absorbent layer being disposed in the receiving space; and
a retention member secured relative to a proximal end of the body member and defining a channel, the channel being configured to releasably receive at least a portion of another medical article.

13. The medical article of claim 12, wherein the body member further comprises a groove generally aligned with a longitudinal axis of the body member so as to provide a flow path across at least a portion of the body member.

14. The medical article of claim 13 further comprising a recess in the body member, the recess being fluidly coupled to the groove.

15. The medical article of claim 13, wherein the groove is disposed on a surface of the absorbent layer.

16. The medical article of claim 12, wherein the retention member is integral with the backing layer.

17. A method comprising:
providing a pad having an absorbent layer and a retention member secured relative to the absorbent layer, the retention member defining a channel; and
placing at least a portion of a medical article within the channel such that the received portion of the medical article is secured relative to the pad.

18. The method of claim 17 further comprising introducing the medical article into the vasculature of a patient such that at least a portion of a volume of blood that passes from the vasculature through the medical article contacts the absorbent layer.

19. The method of claim 18 further comprising removing a medical line from the medical article such that at least a portion of a fluid flowing from the removed medical line contacts the absorbent layer.

20. The method of claim 19 further comprising adhering the pad to a patient.
